(19) United States
(12) Patent Application Publication
(10) Pub. No.: US 2010/0330010 A1
(43) Pub. Date: Dec. 30, 2010

(54) COMPOSITION AND METHOD FOR REDUCING HARMFUL EFFECTS OF ULTRAVIOLET RADIATION IMPINGING ON THE SKIN

(75) Inventor: Peter E. Fuller, Sandy, UT (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: SIMPLURE, LLC, Sandy, UT (US)

(21) Appl. No.: 12/641,046

(22) Filed: Dec. 17, 2009

Related U.S. Application Data
(63) Continuation of application No. 11/211,191, filed on Aug. 25, 2005, now abandoned.
(60) Provisional application No. 60/604,888, filed on Aug. 26, 2004.

(51) Int. Cl. A61K 8/368; A61Q 17/04; A61P 17/02

(52) U.S. Cl. 424/60

(57) ABSTRACT

A composition is provided that is suitable for topical administration for the treatment of skin to reduce the harmful effects of ultraviolet radiation impinging on the skin, the composition comprising acetylsalicylic acid and sodium bicarbonate. Also provided is a method for treating skin to reduce the harmful effects of ultraviolet radiation impinging on the skin comprising topically administering to the skin a therapeutically effective amount of a composition comprising acetylsalicylic acid and sodium bicarbonate. Also provided is an aqueous composition suitable as an additive to a composition of cosmetics comprising acetylsalicylic acid and sodium bicarbonate. Also provided is a method of manufacturing a cosmetic composition, comprising obtaining an aqueous composition that includes acetylsalicylic acid and sodium bicarbonate, and combining the aqueous composition with cosmetic ingredients to create the cosmetic composition.

### RELATED APPLICATIONS

This application is a continuation of U.S. patent application Ser. No. 11/211,191, filed Aug. 25, 2005, and entitled COMPOSITION AND METHOD FOR REDUCING HARMFUL EFFECTS OF ULTRAVIOLET RADIATION IMPINGING ON THE SKIN, which claims priority to U.S. Provisional Patent Application No. 60/604,888, filed Aug. 26, 2004, both of which are incorporated by reference in their entireties. This application is also related to U.S. patent application Ser. No. 10/171,253, filed Jun. 12, 2002, which is incorporated herein by reference in its entirety.

### FIELD OF THE INVENTION

The present invention relates to compositions and methods for reducing the harmful effects, such as sunburn, of solar radiation impinging on the skin.

### BACKGROUND OF THE INVENTION

Although there has been substantial effort in recent years to reduce or eliminate the risk of sunburn (solar erythema) produced by certain wavelengths in the ultraviolet (UV) region of the spectrum, there are still some limitations and disadvantages to many sunscreens and sunblocks. The main objective of a formulation of sunscreen or sunblock has been the prevention of sunburn and other sun damage associated with exposure to solar radiation. Conventional sunscreens typically include those listed in the current version of 37 C.F.R. sctn. 352.10, which is incorporated herein by reference. While these sunscreen active ingredients are often effective, they can be relatively expensive, especially compared to the ingredients of the base or carrier compositions that are used in commercially available sunscreen products.

### BRIEF SUMMARY OF THE INVENTION

The present invention relates to compositions and methods that can be used to reduce sunburn and other skin damage that would otherwise be experienced in response to exposure of the skin to ultraviolet and other solar radiation.

In one aspect of the invention, a composition comprising acetylsalicylic acid and sodium bicarbonate is provided that when topically applied to the skin can reduce the harmful effects of solar radiation impinging on the skin. The compositions are effective for preventing sunburn or other skin damage and are also effective for relieving symptoms of sunburn or other skin damage that has already been experienced.

In another aspect of the invention, there is provided a method for treating skin to reduce the harmful effects of ultraviolet radiation impinging on the skin comprising topically administering to the affected area a therapeutically effective amount of a composition containing acetylsalicylic acid and sodium bicarbonate.

In another aspect of the invention, there is provided an aqueous composition suitable for use as an additive to or a base for a composition of cosmetics, the aqueous composition comprising a solvent, acetylsalicylic acid and sodium bicarbonate.

In another aspect of the invention, there is provided a method of manufacturing a cosmetic composition comprising obtaining an aqueous composition that includes water and, optionally, at least one other solvent; acetylsalicylic acid; and sodium bicarbonate. The aqueous composition is combined with cosmetic ingredients to create the cosmetic composition.

These and other objects and features of the present invention will become more fully apparent from the following description and appended claims, or may be learned by the practice of the invention as set forth hereinafter.

### DETAILED DESCRIPTION OF THE PREFERRED EMBODIMENTS

The methods by which the objects, features and advantages of the present invention are achieved will now be described in more detail. These particulars provide a more precise description of the invention for the purpose of enabling one of ordinary skill in the art to practice the invention, but without limiting the invention to the specific embodiments described.

As used herein, the term “skin irritation” is intended to refer to any condition of the skin causing discomfort, including that caused by burns, such as sunburn, wounds, such as a laceration, insect bites, poisonous plants, and/or allergens. One example of a skin irritation is solar erythema, or sunburn, which can result from prolonged exposure to ultraviolet solar radiation.

As used herein, the term “therapeutically effective amount” is intended to refer to that amount necessary to reduce the effects of ultraviolet radiation impinging on the skin.

As used herein, the term “topically administering” is intended to refer to the direct laying on or spreading of, a composition on epidermal tissue at the affected site of the epidermal tissue.

Existing sunscreens operate by absorbing or reflecting a portion of the ultraviolet solar radiation that would otherwise be absorbed by the skin. It has been discovered that the compositions disclosed herein and in U.S. patent application Ser. No. 10/171,253, filed Jun. 12, 2002, enhance the effectiveness of existing sunscreen compositions. It has also been discovered that the compositions disclosed herein, when topically applied to the skin in therapeutically effective amounts, reduce the degree of solar erythema that would otherwise be experienced in response to prolonged exposure to solar radiation.

In addition, as described in U.S. patent application Ser. No. 10/171,253, filed Jun. 12, 2002, the compositions disclosed herein relieve the symptoms of erythema, including solar erythema. Thus, the compositions disclosed herein can be used to prevent or reduce the onset of solar erythema when applied prior to or during sun exposure. The compositions can further relieve symptoms of solar erythema when applied prior to, during, or after sun exposure.

While conventional sunscreen active ingredients, such as many of those listed in 37 C.F.R. sctn. 352.10, are generally effective in reducing the solar erythema that would otherwise be experienced upon exposure to solar radiation, many such active ingredients are relatively expensive. It has been found that the use of the compositions disclosed herein as part of the base or carrier for any or all of the conventional sunscreen active ingredients enhances the effectiveness of the resulting sunscreen compositions. In addition, many of the compositions disclosed herein are relatively inexpensive compared to conventional sunscreen active ingredients. Moreover, the safety for human use for most of the components of the compositions described herein has already been established. Thus, the compositions of the invention can generally be used as part of the base of a sunscreen composition to enhance the effectiveness of conventional sunscreen active ingredients without safety concerns.

In one aspect of the invention, there is provided a composition for the treatment of skin to reduce the harmful effects of exposure to ultraviolet radiation on the skin, the composition comprising acetylsalicylic acid and sodium bicarbonate.

In another aspect of the invention, there is provided a method for treating skin to reduce the harmful effects of exposure to ultraviolet radiation impinging on the skin comprising topically administering to a treated area of the skin a therapeutically effective amount of a composition containing acetylsalicylic acid and sodium bicarbonate.

In another aspect of the invention, there is provided an aqueous composition suitable as an additive to a composition of cosmetics, the aqueous composition comprising a solvent, acetylsalicylic acid and sodium bicarbonate.

In another aspect of the invention, there is provided a method of manufacturing a cosmetic composition comprising obtaining an aqueous composition that includes water and, optionally, at least one other solvent, acetylsalicylic acid and sodium bicarbonate. The aqueous composition is combined with cosmetic ingredients to create the cosmetic composition.

The composition of the invention can exist in a variety of delivery forms, including, for example, solutions, creams, lotions, emulsions, ointments, aerosols, gels, and solids. Preferably, the composition exists in solution form, wherein the solvent is preferably water. This aqueous composition is preferably of a character that is adapted to be sufficiently thin to allow spray application but not so thin as to flow away from the treated area upon such application. It is preferable to use sterile, deionized water as the solvent, although a less grade of purified water is sufficient for the functional purposes of the invention.

Acetylsalicylic acid is preferably present in the range from about 0.1 to about 10 weight percent based on the final total weight of the aqueous composition after all components have been included in the composition. The weight percent of each component included in the aqueous composition disclosed herein is based on the final total weight of the aqueous composition. Most preferably, the acetylsalicylic acid is present in an amount corresponding to about 2.5 weight percent.

Sodium bicarbonate is preferably present in the range from about 0.1 to about 10 weight percent based on the final total weight of the aqueous composition after all components have been included in the composition. Most preferably, the sodium bicarbonate is present in an amount corresponding to about 2.5 weight percent.

In addition to acetylsalicylic acid and sodium bicarbonate, the composition of the invention can further comprise one or more of the members selected from the group consisting of acidifying agents, alkalizing agents, aerosol propellants, antimicrobial agents, antioxidants, buffering agents, chelating agents, coloring additives, dispersing agents, emollients, emulsifying agents, humectants, fragrances, masking agents, preservatives, sugars, additional sunscreen agents, surfactants, suspending agents, and thickening agents.

Acidifying and alkalizing agents can be added to obtain the desired pH of the composition. Examples of acidifying agents included acetic acid, citric acid, glacial acetic acid, malic acid, and proprionic acid. Examples of alkalizing agent include edetol, potassium carbonate, potassium hydroxide, sodium borate, sodium carbonate, and sodium hydroxide.

Aerosol propellants can be used when the composition is to be administered as an aerosol (solution) under pressure. Examples of aerosol propellants include halogenated hydrocarbons such as dichlorodifluoromethane, dichlorotetrafluoroethane, and trichloromonofluoromethane, nitrogen, and volatile hydrocarbons such as butane, propane, isobutane, or mixtures thereof.

Anti-microbial agents can be used when the area that the composition is to be applied is prone to microbial infection, e.g., by bacteria, fungal, or protozoa. Examples of such agents include benzyl alcohol, chlorobutanol, phenylethyl alcohol, phenylmercuric acetate, potassium sorbate, and sorbic acid, benzoic acid, butyl paraben, ethyl paraben, methyl paraben, propyl pareben, and sodium benzoate.

Antioxidants can be used to protect ingredients of the composition from oxidizing agents that are included within or come in contact with the composition. Examples of antioxidants include water-soluble antioxidants such as ascorbic acid, sodium sulfite, metabisulfite, sodium miosulfite, sodium formaldehyde, sulfoxylate, isoascorbic acid, isoascorbic acid, cysteine hydrochloride, 1,4-diazobicyclo-(2,2,2)-octane, and mixtures thereof. Examples of oil-soluble antioxidants include ascorbyl palmitate, butylated hydroxyanisole, butylated hydroxytoluene, potassium propyl gallate, octyl gallate, dodecyl gallate, phenyl-alpha-napthyl-amine, and tocopherols such as alpha-tocopherol.

Buffering agents can be used to maintain an established pH of the composition. Examples of buffering agents included sodium citrate, calcium acetate, potassium metaphosphate, potassium phosphate monobasic, and tartaric acid.

Chelating agents can be used to maintain the ionic strength of the composition and/or bind to destructive compounds and metals that are included within or come in contact with the composition. Examples of chelating agents included dihydroxy ethyl glycine, citric acid, tartaric acid, edatate dipotassium, edetate disodium, edetic acid, and ethylenediamine tetracetic acid (EDTA) and its salts (e.g., tetrasodium EDTA).

Coloring additives can be used to add color to the composition. Examples of such coloring additives include titanium dioxide, yellow iron oxide, red iron oxide, black iron oxide, caramel, carmine, fluorescein derivatives, methoxsalen, trioxsalen, carbon black, azo dyes, anthraquinone dyes, blue azulenes, guajazulene, chamuzulene, erythrosin, bengal rose, phloxin, cyanosin, daphinin, eosin G, eosin 10B, and Acid Red 51.

Examples of dispersing and suspending agents include quarternium-18 hectorite, polyhydroxy stearic acid, poligeenan and silicon dioxide.

Emollients are agents that soften and smooth the skin. Examples of emollients include hydrocarbon oils and waxes (e.g., natural and synthetic waxes) such as mineral oil, petrolatum, microcrystalline wax, polyethylene, triglyceride esters such as those of castor oil, cocoa butter, safflower oil, cottonseed oil, corn oil, olive oil, cod liver oil, almond oil, avocado oil, palm oil, sesame oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholidds, and sterols.

Emulsifying agents can be used for preparing emulsions of the present invention. Examples of emulsifying agents used for preparing water-in-oil emulsions include cyclomethicone (and) dimethicone copolyol, dimethicone copolyol, cetyl dimethicone copolyol, PEG-30 dipolyhydroxystearate, and PEG-40 sorbitan peroleate. Examples of emulsifying agents used for preparing oil-in-water emulsions of the present invention include glyceryl stearate, PEG-10 a stearate, methyl gluceth sesquisterate, fatty alcohols, and alkyl phenols condensed with ethylene oxide.

Humectants are agents that promote the retention of moisture, e.g., moisturizers. Examples of humectants include sorbitol, matricaria extract, aloe barbadensis gel, glycerin, glycereth 5 lactate, glycereth 7 triacetate, glycereth 7 diisononoate, hexanetriol, hexylene glycol, propylene glycol, dipropylene glycol, alkoxylated glucose, D-panthenol, 1-2-pantandiol, 2-methyl-1,3-propanediol, and derivatives thereof, and hyaluronic acid.

Examples of fragrances include peppermint, rose oil, rose water, aloe vera, clove oil, menthol, camphor, eucalyptus oil, and other plant extracts. Certain fragrances may require a solubilizer, e.g., PPG-5-ceteareth-20. To eliminate certain odors from compositions, masking agents may be used. An example of a masking agent includes ethylene brassylate.

Preservatives can be used to protect the composition from degradation. Examples of preservatives include liquipar oil, phenoxyethanol, methyl paraben, propyl paraben, butyl paraben, isopropyl paraben, isobutyl paraben, dieizolidinyl urea, imidazolidinyl urea, diazolindyl urea, benzalkonium chloride, benzethonium chloride, phenol, and mixtures thereof (e.g., liquipar oil).

Examples of sugars include monosaccharides, disaccharides, and polysaccharides such as glucose, xylose, fructose, reose, ribose, pentose, arabinose, allose, tallose, altrose, mannose, galactose, lactose, sucrose, erythrose, glyceraldehyde, or any combination thereof.

Conventional sunscreen agents are agents used to block or reduce the amount of ultraviolet radiation impinging on the skin (e.g., by absorption, scattering, and reflection of the ultraviolet radiation). Examples of sunscreen agents include both organic compounds and their salts such as octyl methoxycinnamate, octyl salicylate, benzophenone-3 homosalate, octocrylate, avobenzone, and menthyl anthranilate, as well as inorganic particulate materials such as zinc oxide, silica, iron oxide, titanium dioxide, and 2-ethyl-hexyl-p-methoxycinnamate.

Surfactants are agents used to stabilize multi-component compositions, e.g., used as wetting agents, antifoam agents, emulsifiers, dispersing agents, and penetrants. Examples of surfactants include methyl gluceth 20, decyl polyglucoside, lapyrium chloride, laureth 4, laureth 9, monoethanolamine, nonoxynol 4, nonoxynol 9, nonoxynol 10, nonoxynol 15, nonoxynol 30, poloxalene, polyoxyl 8, 40, and 50 stearate, polysorbate 20, polysorbate 40, polysorbate 60, polysorbate 65, polysorbate 80, and polysorbate 85, sodium lauryl sulfate, sorbitan and its derivatives.

Preferably, the aqueous composition of the invention includes a humectant, such as glycerin. The humectant tends to reduce the tackiness produced by the aqueous composition after it has been applied and has dried on the skin. The humectant is present in an amount such as to produce an aqueous composition that is capable of being substantially non-tacky after drying on the skin. Preferably, the humectant is present in the range from about 0.1 to about 20 weight percent based on the final total weight of the water-based formulation after all components have been included in the aqueous composition. Most preferably, the humectant is present in an amount corresponding to about 5 weight percent.

It is to be understood that the above components can be mixed and/or blended together using standard techniques well known in the art to achieve the inventive composition. It is further to be understood that the order of addition of each component is not important in preparing the composition.

The aqueous composition of the invention can be sealed in an appropriate carrier or container. Such a carrier or container may be a tube, a bottle, a single-dose packet or the like. Preferably, the container is a pump spray bottle. The tube or bottle can also be collapsible so as to permit convenient application of a relatively large quantity of the aqueous composition to a sunburned area.

In view of the fact that acetylsalicylic acid is highly insoluble in water, aqueous compositions that include acetylsalicylic acid and sodium bicarbonate generally exist in a state in which most of the acetylsalicylic acid is a precipitate that settles to the bottom of a volume of the composition. Thus, during storage, much of the acetylsalicylic acid is effectively isolated from the sodium bicarbonate and can be maintained for prolonged periods without experiencing reaction with the sodium bicarbonate. At the time of application, when the composition is agitated, shaken, or otherwise mixed, the acetylsalicylic acid and the sodium bicarbonate are permitted to react, thereby generating the carbon dioxide and other products of the reaction.

Furthermore, the skin can be treated to reduce the harmful effects of exposure to ultraviolet radiation with the aqueous composition disclosed herein without producing the undesirable side-effects, such as tackiness, that are frequently encountered, for example, with a petroleum-based composition. In addition, the aqueous composition disclosed herein does not produce a residue that may need to be subsequently removed from a potentially sensitive sunburned area.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

### Experimental Data

Clinical trials have been performed using a composition that includes sodium bicarbonate and acetylsalicylic acid. Table 1 sets forth the results of a representative clinical trial. In this experiment, five subjects having skin type II were treated with the composition using a final monograph methodology. In the experiment, a composition (the “test composition”) formulated according to the invention as disclosed herein was mixed with water and applied to a 2 cm.sup.2 patch of skin on the back of each subject (a “test patch”) at a concentration of 2 mg/cm.sup.2. A control sunscreen composition (the “control composition”) with a nominal SPF value of 4.0 was also applied to a comparable 2 cm.sup.2 patch of skin on the back of each subject (a “control patch”).

After the compositions were allowed to dry for 15 minutes, the subjects were irradiated using a solar simulator and subjected to a minimal erythemal dose. The minimal erythemal dose was applied to the test patch and the control patch. The minimal erythemal dose was also applied to a comparable patch of skin on the back of the each subject (an “untreated patch”) that had not received either the composition formulated according to the invention or the control composition.

After twenty-four hours, the subjects were examined to determine the extent of erythema exhibited on each of the three patches. The untreated patch was used as a baseline to calculate the SPF rating assigned to the control composition and the test composition. In particular, the untreated patch established a normalized value of 1.0, which was used as a baseline for evaluating the SPF effectiveness of the control composition and the test composition. The experiment yielded the following results:

Individual SPF Values

| | | | | 8% HMS | |
|---|---|---|---|---|---|
| Subject | CPTC # | Skin Type | Age/Sex | Standard | PJK #1 |
| 1) S D | 42555 | II | 19/F | 4.0 | 4.0 |
| 2) D D | 1830 | II | 43/M | 4.4 | 3.2 |
| 3) R C | 27398 | II | 40/M | 5.0 | 3.2 |
| 4) V S | 11356 | II | 55/F | 5.0 | 6.3 |
| 5) J N | 40840 | II | 39/M | 5.0 | 3.6 |

| | | |
|---|---|---|
| Average SPN (N = 5) | 4.8 | 4.06 |
| Standard Deviation | 0.46 | 1.30 |
| Standard Error | 0.21 | 0.58 |
| t (one tail) | 2.132 | 2.132 |
| A | 0.44 | 1.24 |
| SPF Label | 4.24 | 2.82 |

In Table 1, the column designated as “8% HMS Standard” sets forth the personal SPF values assigned to the control composition for each of the subjects in the experiment. The column designated as “PJK #1” sets forth the personal SPF values assigned to the test composition for each of the subjects. Table 1 also shows the average SPF value of 4.68 associated with the control composition in this test and the average SPF value of 4.06 of the test composition. In contrast, a composition that exhibits no reduction or prevention of the incidence of erythema would have an SPF value of 1.0. This experiment indicates that the test composition was nearly as effective at preventing or reducing the incidence of erythema as was the control composition.

What is claimed is:

1. An aqueous composition suitable for topical administration for the treatment of skin to reduce harmful effects of ultraviolet radiation impinging on skin, the aqueous composition consisting of:
water and, optionally, at least one other solvent;
acetylsalicylic acid;
sodium bicarbonate; and
optionally a humcetant.

2. The aqueous composition of claim 1 wherein acetylsalicylic acid is present in about 2.5 weight percent.

3. The aqueous composition of claim 1 wherein sodium bicarbonate is present in about 2.5 weight percent.

4. A method for reducing harmful effects of ultraviolet radiation impinging on skin comprising:
topically administering an aqueous composition to the skin of an individual, wherein the aqueous composition consists of:
water and, optionally, at least one other solvent;
acetylsalicylic acid;
sodium bicarbonate; and
optionally a humectant.

5. The method of claim 4 wherein acetylsalicylic acid is present in about 2.5 weight percent.

6. The method of claim 4 wherein sodium bicarbonate is present in about 2.5 weight percent.

7. The method of claim 4, wherein the method comprises topically administering the aqueous composition to a burn in the skin of the individual.

8. The method of claim 4, wherein the method comprises topically administering the aqueous composition to sunburned skin of the individual.

9. A method for reducing harmful effects of ultraviolet radiation impinging on skin, comprising:
obtaining an aqueous composition that consists of:
water that operates as a solvent in the aqueous composition;
glycerin that operates as a humectant in the aqueous composition;
acetylsalicylic acid in a range of 0.1% to 10% by weight with respect to the weight of the aqueous composition;
sodium bicarbonate in a range of 0.1% to 10% by weight with respect to the weight of the aqueous composition and,
topically administering a therapeutically effective amount of the aqueous composition to the skin of an individual.

10. The method of claim 9 wherein acetylsalicylic acid is present in about 2.5 weight percent.

11. The method of claim 9 wherein sodium bicarbonate is present in about 2.5 weight percent.
